(19) United States
(12) Patent Application Publication
(10) Pub. No.: US 2018/0126088 A1
(43) Pub. Date: May 10, 2018

(54) Rotary Sensor Arrangement for Drug Delivery Device

(71) Applicant: Novo Nordisk A/S, Bagsvaerd (DK)

(72) Inventors: Bo Radmer, Hilleroed (DK); Roger Harrington, Skaevinge (DK); Nikolai Byskov, Noerrebro (DK); Bennie Peder Smiszek Pedersen, Haslev (DK)

(73) Assignee: Novo Nordisk A/S, Bagsvaerd (DK)

(21) Appl. No.: 15/571,405

(22) PCT Filed: May 11, 2016
(86) PCT No.: PCT/EP2016/060546

§ 371 Date: Nov. 2, 2017

(30) Foreign Application Priority Data
May 12, 2015 (EP) 15167274.8

(51) Int. Cl. A61M 5/315; G01D 5/252

(52) U.S. Cl. CPC A61M 5/31553; A61M 5/3158; H01H 19/00; A61M 5/31583; A61M 5/31593; G01D 5/252

(57) ABSTRACT

A rotary sensor assembly comprises an indicator member adapted to rotate and having first and second axial positions, input means adapted to be actuated by movement of the indicator member, and a processor adapted to receive input from the input means. The indicator member comprises a plurality of actuator structures, and the input means comprises one or more switches adapted to be actuated by an actuator structure. Zero or more switches is/are actuated when the indicator member is moved from the first to the second axial position, this corresponding to a first switch pattern, and zero or more switches is/are actuated when the indicator member is moved from the second to the second axial position, this corresponding to a second switch pattern. Based on input from one or more switches corresponding to the first and second switch patterns, the processor is adapted to determine rotational movement of the indicator member.

The present invention generally relates to sensor and trigger arrangements suitable for use in a medical device. In a specific aspect the medical device comprises indicator means configured to display information relating to an expelled dose of drug.

### BACKGROUND OF THE INVENTION

In the disclosure of the present invention reference is mostly made to drug delivery devices comprising a threaded piston rod, such devices being used e.g. in the treatment of diabetes by delivery of insulin, however, this is only an exemplary use of the present invention.

Drug Injection devices have greatly improved the lives of patients who must self-administer drugs and biological agents. Drug Injection devices may take many forms, including simple disposable devices that are little more than an ampoule with an injection means or they may be durable devices adapted to be used with pre-filled cartridges. Regardless of their form and type, they have proven to be great aids in assisting patients to self-administer injectable drugs and biological agents. They also greatly assist care givers in administering injectable medicines to those incapable of performing self-injections.

The typical diabetes patient will require injections of insulin several times during the course of a week or a day. For other types of drug the intervals between drug deliveries may be shorter or longer. However, typical injection devices do not address the problem of a user not remembering when the last injection was administered.

Even shortly after administering a dose of insulin, the user now and then will be in doubt as to whether he actually carried out an injection or not. This could be after minutes or even hours after the intended time for performing an administration. Thus, there exist the potential hazard that the patient chooses not to take his or her medication or that it is taken twice.

Some prior art devices, such as the electronic drug delivery device disclosed in WO 97/30742, are provided with an electronic monitoring system adapted to automatically start an electronic timer when a selected dose is expelled and to show the progress in time on an electronic display. Such an injection device generally provides a satisfactorily solution to the problem addressed above. However, for cheaper and simpler devices such as disposable drug delivery devices, i.e. so-called pre-filled devices, the incorporation of this kind of electronics would normally not be economically viable.

Addressing this issue, WO 99/43283 discloses a timer device which is intended to be used with pre-filled injection pens, where the timer device is configured for releasable attachment to the pre-filled pen so that the timer device can be removed from a pen once it is ready for disposal and be attached to a new pen. The timer device is configured to detect when an injection is performed and to communicate this via indicator lights that remains turned on for a certain time period from the administration of the dose. WO 2010 discloses an add-on module for a reusable or disposable drug pen device, the module being adapted to determine the size of a set and/or expelled dosage of drug.

As an alternative to using an add-on device which has to be removed and attached each time the user has emptied a pre-filled drug delivery device, WO 2010/023303 discloses a drug delivery device provided with a non-electronic time delay indicator integrated in the proximal push button, the arrangement providing a simple and cost-effective solution allowing the indicator to be provided as an integral part of a pre-filled device.

Although the above-described two alternatives to a build-in electronic timer device may provide useful solutions to some users, an electronic timer device which could be provided as an integral part of a relatively inexpensive drug delivery device, either durable or disposable, would be desirable. Such a design would be more user-friendly as compared to an add-on solution just as the electronics per se would allow greater freedom to design the user interface, e.g. display design and control thereof. Such an electronic timer device for a drug delivery device would comprise the electronic circuitry per se, e.g. processor, display and power source, as well as a trigger or switch arrangement for initiating the timer functionality. In addition to the timer functionality the timer device may be provided with the ability to detect the size of a set and/or expelled dose, thereby providing a dose logging functionality.

Having regard to the above, it is an object of the present invention to provide a switch arrangement suitable for use in a drug delivery device and adapted to be actuated by movement of an indicator member. The switch arrangement may be in the form of a trigger assembly. The arrangement may be used independently or in combination with other trigger or switch arrangements. It is a further object to provide a switch arrangement which may also serve as a rotary sensor. The switch arrangement should be reliable in use and designed for cost-effective manufacturing.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention a rotary sensor assembly is provided, comprising an indicator member adapted to rotate and having a rotationally locked first axial position and a rotationally free second axial position, input means adapted to be actuated, directly or indirectly, by movement of the indicator member, and a processor adapted to receive input from the input means. The indicator member comprises a plurality of actuator structures, and the input means comprises one or more switches adapted to be actuated by an actuator structure. Zero or more switches is/are actuated when the indicator member is moved from the first to the second axial position, this corresponding to a first switch pattern, and zero or more switches is/are actuated when the indicator member is moved from the second to the second axial position, this corresponding to a second switch pattern. In such an arrangement the processor is adapted to determine rotational movement of the indicator member based on input from the one or more switches corresponding to the first and second switch patterns.

By the above arrangement a rotary sensor is provided which cost-effectively can be incorporated in an assembly comprising a rotational member adapted to move between axial positions. The indicator member may be adapted to rotate in increments, the processor being adapted to determine incremental movement of the indicator member based on input from the one or more switches corresponding to the first and second switch patterns.

As will be described below for an exemplary embodiment, in a simple embodiment one switch can provide the described functionality. More specifically, if a sensor assembly is provided with a single switch which initially is closed and an even number of increment is expelled then this switch may not be actuated as the actuator structures move back and forth resulting in zero switches being actuated. If an odd number of increments are expelled then this switch would be actuated only once as the actuator structures move back and forth. As appears, actuation of such a sensor system would result in either a single or no input to the electronic circuitry.

Thus, to ensure “positive” and safe detection of movement, in an exemplary embodiment at least one switch is actuated when the indicator member is moved from the first to the second axial position, this corresponding to a first switch pattern, and at least one switch is actuated when the indicator member is moved from the second to the first axial position, this corresponding to a second switch pattern. In this way positive and safe detection of indicator movement can be ensured as an on-off input is provided for all incremental rotational movements including no rotation.

The actuator structures and one or more switches may be arranged to provide first and second switch patterns allowing the processor to determine whether the indicator member has rotated corresponding to an even or odd number of increments.

Correspondingly, in an exemplary embodiment the rotary sensor assembly comprises first and second switches, the actuator structures being arranged on the indicator member such that (i) for a given rotational position the first switch only is actuated by an actuator structure when the indicator member is moved from the first to the second axial position, this corresponding to the first switch pattern, and (ii) for a rotational movement of an odd number of increments the second switch only is actuated when the indicator member is moved from the second to the first axial position, this corresponding to the second switch pattern.

In a further aspect of the invention a drug delivery device is provided comprising a housing having an exterior surface, a rotary sensor assembly as described above, a drug-filled cartridge or means for receiving a drug-filled cartridge, the cartridge comprising an outlet and an axially displaceable piston, drug expelling means and a sensor system. The drug expelling means comprises dose setting means allowing a user to set a dose amount of drug to be expelled, a piston rod adapted to engage and axially move the piston to thereby expel an amount of drug from the cartridge through the outlet, and the indicator member of the rotary sensor assembly. The sensor system comprises the above-described input means adapted to be actuated, directly or indirectly, by movement of the indicator member, the processor adapted to receive input from the input means, and an energy source. In such a drug delivery device the indicator member is arranged to move during expelling of a dose, the indicator member is in the first axial position when the drug expelling means is in a dose setting state, the indicator member is in the second axial position when the drug expelling means is in an expelling state, and the indicator member is adapted to rotate during expelling of a dose corresponding to a set dose. Further, the indicator member comprises a plurality of actuator structures, and the input means comprises one or more switches adapted to be actuated by an actuator structure. The switches are arranged such that zero or more switches is/are actuated when the indicator member is moved from the first to the second axial position, this corresponding to a first switch pattern, and such that zero or more switches is/are actuated when the indicator member is moved from the second to the second axial position, this corresponding to a second switch pattern. The processor is then adapted to determine rotational movement of the indicator member based on input from the one or more switches corresponding to the first and second switch patterns.

By this arrangement a drug delivery device is provided in which the above-described sensor assembly is cost-effectively incorporated.

In an exemplary embodiment the dose setting means is adapted to set a dose in increments, the amount of rotation of the indicator member during expelling of a dose corresponds to a number of increments, and the processor is adapted to determine incremental movement of the indicator member based on input from the one or more switches corresponding to the first and second switch patterns. The actuator structures and the one or more switches may arranged to provide first and second switch patterns allowing the processor to determine whether the indicator member during expelling of a dose has rotated corresponding to a an even or odd number of increments.

In a further exemplary embodiment the drug delivery device comprises first and second switches, the actuator structures being arranged on the indicator member such that (i) for a given rotational position the first switch only is actuated by an actuator structure when the indicator member is moved from the first to the second axial position, this corresponding to the first switch pattern, and (ii) for a rotational movement of an odd number of increments the second switch only is actuated when the indicator member is moved from the second to the first axial position, this corresponding to the second switch pattern.

The drug delivery device may further comprise a second indicator member arranged to move during expelling of a dose, as well as a second sensor system comprising second input means adapted to be actuated, directly or indirectly, by movement of the second indicator member, the processor being adapted to receive input from the second input means.

The second indicator member may be adapted to rotate from a set position corresponding to a set dose amount and to an end-of-dose position in which the set dose has been expelled. In such an arrangement the second indicator member has a first axial position when the drug expelling means is in a dose setting state, and a second axial position when the drug expelling means is in an expelling state, and the second input means is actuated when the second indicator member has reached the end-of-dose position when the second indicator member is in the second axial position. Alternatively, the second indicator member may be adapted to rotate from an initial position to an end-of-dose position in which the set dose has been expelled, the amount of rotation corresponding to the expelled dose amount.

By such combined arrangements a drug delivery device may be provided which cost-effectively can be adapted to cope with issues based on tolerances and slack in the dose setting and expelling mechanism when detecting an end-of-dose event.

In an exemplary embodiment the drug delivery device comprises a display adapted to display a time parameter, the processor being adapted to, based on input from the input means, control the display to display a time parameter related to the time the input means was actuated. The drug delivery device may comprise a flexible sheet on which is formed or mounted the display adapted to display a time parameter, the processor, and the energy source, the flexible sheet being mounted at least in part to the exterior of the housing. One or more of the display, processor, and energy source may be in the form of printed electronics.

In a further aspect of the invention a drug delivery device is provided comprising a drug-filled cartridge or means for receiving a drug-filled cartridge, the cartridge comprising an outlet and an axially displaceable piston, drug expelling means, and a sensor system. The drug expelling means comprises dose setting means allowing a user to set a dose amount of drug to be expelled a piston rod adapted to engage and axially move the piston to thereby expel an amount of drug from the cartridge through the outlet, and an indicator member arranged to move during expelling of a dose. The sensor system comprises input means adapted to be actuated, directly or indirectly, by movement of the indicator member, a processor adapted to receive input from the input means, and an energy source. In such an arrangement the indicator member is adapted to rotate from a set position corresponding to a set dose amount and to an end-of-dose position in which the set dose has been expelled, the indicator member having a first axial position when the drug expelling means is in a dose setting state, and a second axial position when the drug expelling means is in an expelling state. The input means is actuated when the indicator member has reached the end-of-dose position when the indicator member is in the second axial position.

By the above arrangement an end-of-dose sensor is provided which cost-effectively can be incorporated in an assembly comprising a rotational member adapted to move between axial positions.

Alternatively, the indicator member may be adapted to rotate during expelling from an initial position to an end-of-dose position in which the set dose has been expelled, the amount of rotation corresponding to the expelled dose amount.

In an exemplary embodiment the dose setting means is adapted to set a dose in increments and the drug delivery device comprises a second indicator member and second input means adapted to be actuated, directly or indirectly, by movement of the second indicator member. The amount of rotation of the indicator members during expelling of a dose corresponds to a number of increments, and the second indicator member and second input means are adapted to provide input allowing the processor to determine whether the second indicator member during expelling of a dose has rotated corresponding to an even or odd number of increments.

The drug delivery device may further comprise a display, the processor being adapted to control the display to display a time parameter indicating the time when the input means was actuated, or a time parameter indicating the time since the input means was actuated.

The above-described input means and actuator structures may for example be in the form of a mechanically actuated switch assembly in which a contact or switch is opened or closed by moving a switch structure by means of a structure arranged on or formed with the indicator member, e.g. a protrusion moving a flexible contact finger to open or close a contact.

The switch assembly may be in the form of a laminate comprising a flexible substrate on which a number of contact pads are formed, and a flexible metal sheet forming a number of flexible contact fingers, each finger comprising a contact point which by an actuation structure can be moved into and out of contact with a corresponding contact pad to thereby close and open a switch.

Alternatively, the input means may be in the form of an electric switch assembly in which a pair of conductors is actuated by being electrically connected/dis-connected, and the switch thereby closed/opened, by an indicator structure in the form of a conducting structure arranged on the moving indicator member.

The indicator member comprises a plurality of actuator structures, and the input means comprises one or more switches adapted to be actuated by an actuator structure.

As used herein, the term “drug” is meant to encompass any drug-containing flowable medicine capable of being passed through a delivery means such as a hollow needle in a controlled manner, such as a liquid, solution, gel or fine suspension. Representative drugs include pharmaceuticals such as peptides, proteins, and hormones, biologically derived or active agents, hormonal and gene based agents, nutritional formulas and other substances in both solid (dispensed) or liquid form. In the description of the exemplary embodiments reference will be made to the use of insulin. Other specific drugs could be growth hormone and drugs for the treatment of haemophilia and inflammation.

As used herein, the term “insulin” is meant to encompass any drug-containing flowable medicine capable of being passed through a delivery means such as a cannula or hollow needle in a controlled manner, such as a liquid, solution, gel or fine suspension, and which has a blood glucose controlling effect, e.g. human insulin and analogues thereof as well as non-insulins such as GLP-1 and analogues thereof.

### BRIEF DESCRIPTION OF THE DRAWINGS

In the following exemplary embodiments of the invention will be described with reference to the drawings, wherein

FIG. 1A shows a pen device,

FIG. 1B shows the pen device of FIG. 1A with the pen cap removed,

FIG. 2 shows in an exploded view the components of the pen device of FIG. 1A,

FIGS. 3A and 3B show in sectional views an expelling mechanism in two states,

FIG. 4 shows a switch and sensor assembly implemented in a drug delivery device of the general design shown in FIG. 2,

FIG. 5 shows the combined switch assembly of FIG. 4 in greater detail,

FIG. 6 shows a modified ratchet tube of the type shown in FIG. 2,

FIG. 7 shows in part a further embodiment a switch and sensor assembly implemented in a drug delivery device of the general design shown in FIG. 2,

FIG. 8A shows the switch and sensor assembly of FIG. 7 together with further components in an assembled state,

FIG. 8B shows the switch and sensor assembly of FIG. 8A in a perspective view,

FIGS. 9A-9D shows the assembly of FIG. 8A in different operational states in combination with a perspective view of a portion of the assembly,

FIG. 10 shows in table form for the assembly in FIG. 8 different switch states in accordance with different user operation sequences,

FIG. 11 shows a further sensor assembly,

FIG. 12 shows in part a yet further embodiment a switch and sensor assembly implemented in a drug delivery device of the general design shown in FIG. 2,

FIGS. 13.1-13.7 show different states of use of a drug delivery pen with an electronic label.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessary can be assembled to provide a unitary or functional assembly during a given assembly procedure but is merely used to describe components grouped together as being functionally more closely related.

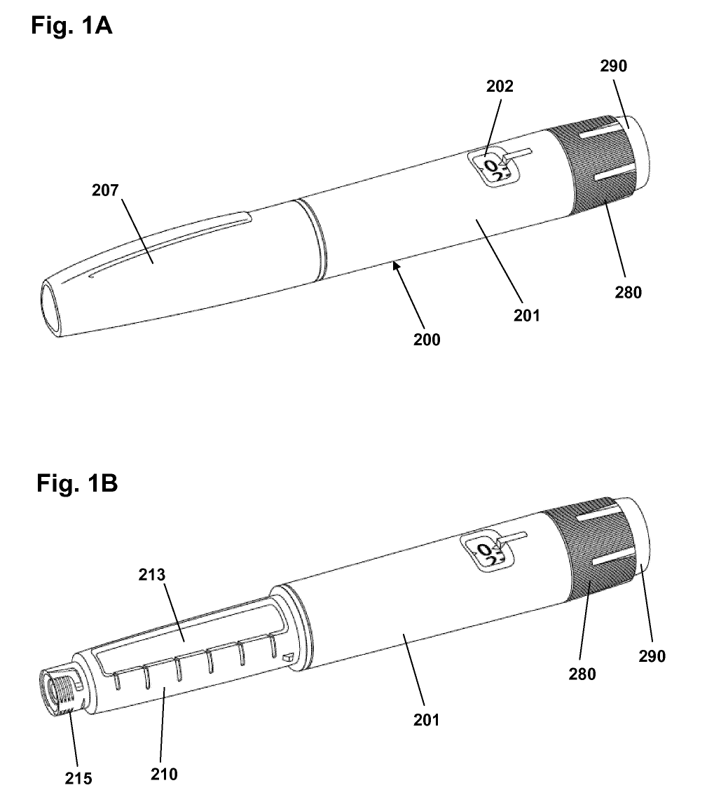

Before turning to embodiments of the present invention per se, an example of a pre-filled drug delivery will be described, such a device providing the basis for the exemplary embodiments of the present invention. Although the pen-formed drug delivery device 200 shown in FIG. 1 may represent a “generic” drug delivery device, the actually shown device is a FlexTouch® pre-filled drug delivery pen as manufactured and sold by Novo Nordisk A/S, Bagsværd, Denmark.

The pen device 200 comprises a cap part 207 and a main part having a proximal body or drive assembly portion with a housing 201 in which a drug expelling mechanism is arranged or integrated, and a distal cartridge holder portion in which a drug-filled transparent cartridge 213 with a distal needle-penetrable septum is arranged and retained in place by a non-removable cartridge holder attached to the proximal portion, the cartridge holder having openings allowing a portion of the cartridge to be inspected as well as distal coupling means 215 allowing a needle assembly to be releasably mounted. The cartridge is provided with a piston driven by a piston rod forming part of the expelling mechanism and may for example contain an insulin, GLP-1 or growth hormone formulation. A proximal-most rotatable dose setting member 280 serves to manually set a desired dose of drug shown in display window 202 and which can then be expelled when the button 290 is actuated. Depending on the type of expelling mechanism embodied in the drug delivery device, the expelling mechanism may comprise a spring as in the shown embodiment which is strained during dose setting and then released to drive the piston rod when the release button is actuated. Alternatively the expelling mechanism may be fully manual in which case the dose member and the actuation button moves proximally during dose setting corresponding to the set dose size, and then is moved distally by the user to expel the set dose, e.g. as in a FlexPen® manufactured and sold by Novo Nordisk A/S.

Although FIG. 1 shows a drug delivery device of the pre-filled type, i.e. it is supplied with a pre-mounted cartridge and is to be discarded when the cartridge has been emptied, in alternative embodiments the drug delivery device may be designed to allow a loaded cartridge to be re-placed, e.g. in the form of a “rear-loaded” drug delivery device in which the cartridge holder is adapted to be removed from the device main portion, or alternatively in the form of a “front-loaded” device in which a cartridge is inserted through a distal opening in the cartridge holder which is non-removable attached to the main part of the device.

As the invention relates to electronic circuitry adapted to be incorporated in and interact with a drug delivery device, an exemplary embodiment of such a device will be described for better understanding of the invention.

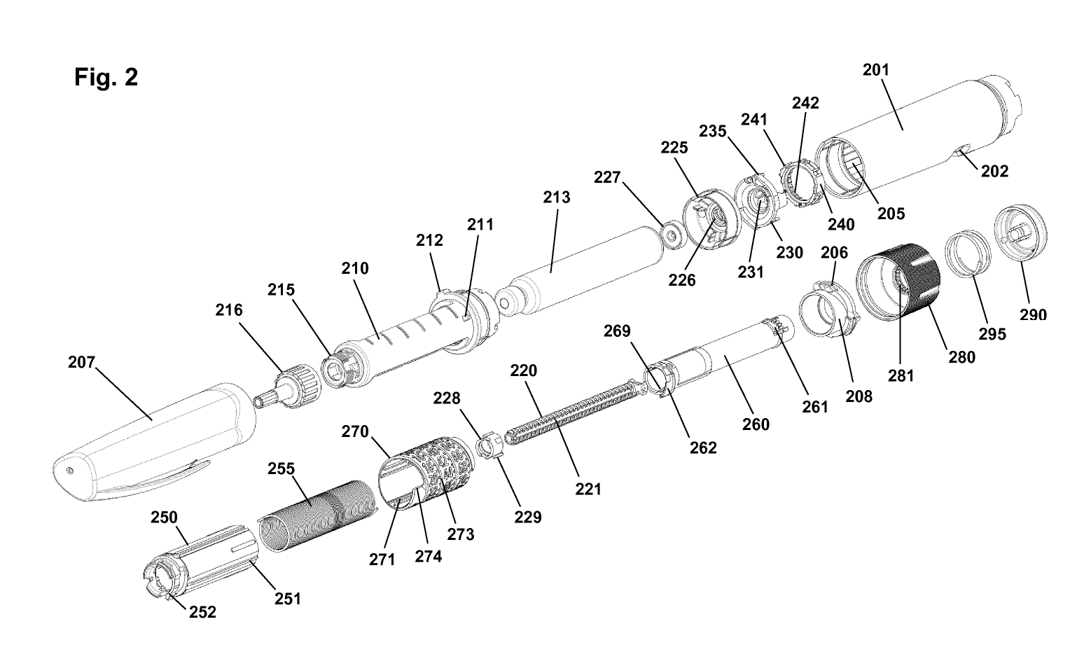

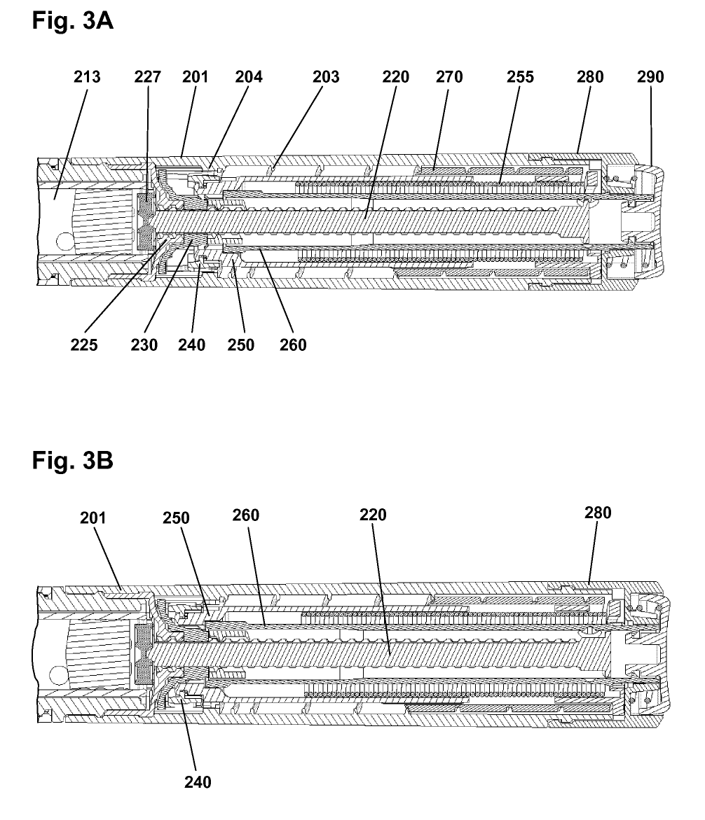

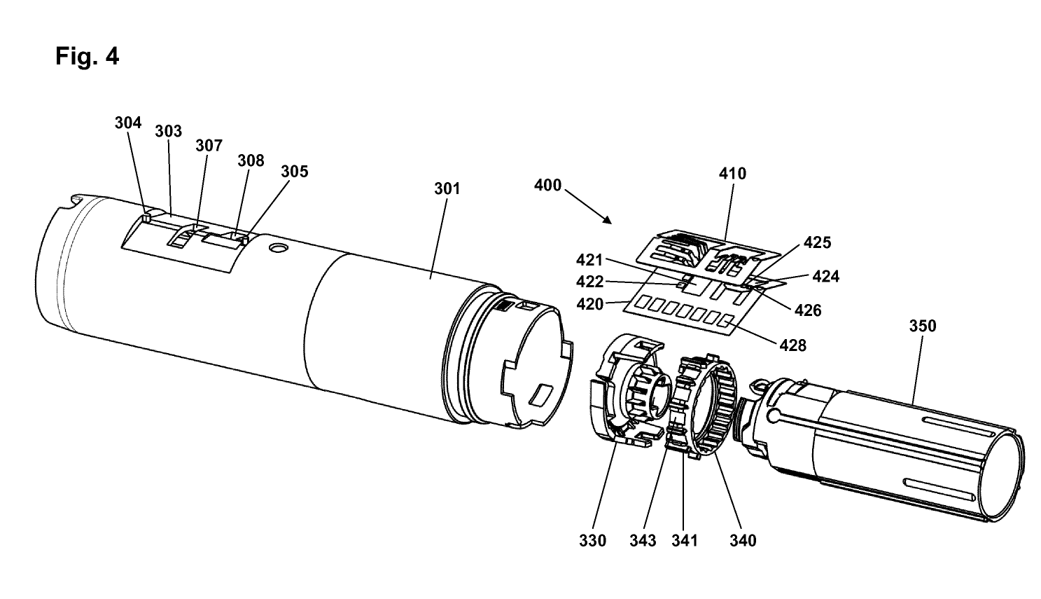

FIG. 2 shows an exploded view of the pen-formed drug delivery device 200 shown in FIG. 1. More specifically, the pen comprises a tubular housing 201 with a window opening 202 and onto which a cartridge holder 210 is fixedly mounted, a drug-filled cartridge 213 being arranged in the cartridge holder. The cartridge holder is provided with distal coupling means 215 allowing a needle assembly 216 to be releasable mounted, proximal coupling means in the form of two opposed protrusions 211 allowing a cap 207 to be releasable mounted covering the cartridge holder and a mounted needle assembly, as well as a protrusion 212 preventing the pen from rolling on e.g. a table top. In the housing distal end a nut element 225 is fixedly mounted, the nut element comprising a central threaded bore 226, and in the housing proximal end a spring base member 208 with a central opening is fixedly mounted. A drive system comprises a threaded piston rod 220 having two opposed longitudinal grooves and being received in the nut element threaded bore, a ring-formed piston rod drive element 230 rotationally arranged in the housing, and a ring-formed clutch element 240 which is in rotational engagement with the drive element (see below), the engagement allowing axial movement of the clutch element. The clutch element is provided with outer spline elements 241 adapted to engage corresponding splines 204 (see FIG. 4B) on the housing inner surface, this allowing the clutch element to be moved between a rotationally locked proximal position, in which the splines are in engagement, and a rotationally free distal position in which the splines are out of engagement. As just mentioned, in both positions the clutch element is rotationally locked to the drive element. The drive element comprises a central bore with two opposed protrusions 231 in engagement with the grooves on the piston rod whereby rotation of the drive element results in rotation and thereby distal axial movement of the piston rod due to the threaded engagement between the piston rod and the nut element. The drive element further comprises a pair of opposed circumferentially extending flexible ratchet arms 235 adapted to engage corresponding ratchet teeth 205 arranged on the housing inner surface. The drive element and the clutch element comprise cooperating coupling structures rotationally locking them together but allowing the clutch element to be moved axially, this allowing the clutch element to be moved axially to its distal position in which it is allowed to rotate, thereby transmitting rotational movement from the dial system (see below) to the drive system. The interaction between the clutch element, the drive element and the housing will be shown and described in greater detail with reference to FIGS. 3A and 3B.

On the piston rod an end-of-content (EOC) member 228 is threadedly mounted and on the distal end a washer 227 is rotationally mounted. The EOC member comprises a pair of opposed radial projections 229 for engagement with the reset tube (see below).

The dial system comprises a ratchet tube 250, a reset tube 260, a scale drum 270 with an outer helically arranged row of dose numerals, a user-operated dial member 280 for setting a dose of drug to be expelled, a release button 290 and a torque spring 255 (see FIG. 3). The reset tube is mounted axially locked inside the ratchet tube but is allowed to rotate a few degrees (see below). The reset tube comprises on its inner surface two opposed longitudinal grooves 269 adapted to engage the radial projections 229 of the EOC member, whereby the EOC can be rotated by the reset tube but is allowed to move axially. The clutch element is mounted axially locked on the outer distal end portion of the ratchet tube 250, this providing that the ratchet tube can be moved axially in and out of rotational engagement with the housing via the clutch element. The dial member 280 is mounted axially locked but rotationally free on the housing proximal end, the dial ring being under normal operation rotationally locked to the reset tube (see below), whereby rotation of dial ring results in a corresponding rotation of the reset tube and thereby the ratchet tube. The release button 290 is axially locked to the reset tube but is free to rotate. A return spring 295 provides a proximally directed force on the button and the thereto mounted reset tube. The scale drum 270 is arranged in the circumferential space between the ratchet tube and the housing, the drum being rotationally locked to the ratchet tube via cooperating longitudinal splines 251, 271 and being in rotational threaded engagement with the inner surface of the housing via cooperating thread structures 203, 273, whereby the row of numerals passes the window opening 202 in the housing when the drum is rotated relative to the housing by the ratchet tube. The torque spring is arranged in the circumferential space between the ratchet tube and the reset tube and is at its proximal end secured to the spring base member 208 and at its distal end to the ratchet tube, whereby the spring is strained when the ratchet tube is rotated relative to the housing by rotation of the dial member. A ratchet mechanism with a flexible ratchet arm 252 is provided between the ratchet tube and the clutch element, the latter being provided with an inner circumferential teeth structures 242, each tooth providing a ratchet stop such that the ratchet tube is held in the position to which it is rotated by a user via the reset tube when a dose is set. In order to allow a set dose to be reduced a ratchet release mechanism 262 is provided on the reset tube and acting on the ratchet tube, this allowing a set dose to be reduced by one or more ratchet increments by turning the dial member in the opposite direction, the release mechanism being actuated when the reset tube is rotated the above-described few degrees relative to the ratchet tube.

Having described the different components of the expelling mechanism and their functional relationship, operation of the mechanism will be described next with reference mainly to FIGS. 3A and 3B.

The pen mechanism can be considered as two interacting systems, a dose system and a dial system, this as described above. During dose setting the dial mechanism rotates and the torsion spring is loaded. The dose mechanism is locked to the housing and cannot move. When the push button is pushed down, the dose mechanism is released from the housing and due to the engagement to the dial system, the torsion spring will now rotate back the dial system to the starting point and rotate the dose system along with it.

The central part of the dose mechanism is the piston rod 220, the actual displacement of the plunger being performed by the piston rod. During dose delivery, the piston rod is rotated by the drive element 230 and due to the threaded interaction with the nut element 225 which is fixed to the housing, the piston rod moves forward in the distal direction. Between the rubber piston and the piston rod, the piston washer 227 is placed which serves as an axial bearing for the rotating piston rod and evens out the pressure on the rubber piston. As the piston rod has a non-circular cross section where the piston rod drive element engages with the piston rod, the drive element is locked rotationally to the piston rod, but free to move along the piston rod axis. Consequently, rotation of the drive element results in a linear forwards movement of the piston. The drive element is provided with small ratchet arms 234 which prevent the drive element from rotating clockwise (seen from the push button end). Due to the engagement with the drive element, the piston rod can thus only move forwards. During dose delivery, the drive element rotates anti-clockwise and the ratchet arms 235 provide the user with small clicks due to the engagement with the ratchet teeth 205, e.g. one click per unit of insulin expelled.

Turning to the dial system, the dose is set and reset by turning the dial member 280. When turning the dial, the reset tube 260, the EOC member 228, the ratchet tube 250 and the scale drum 270 all turn with it. As the ratchet tube is connected to the distal end of the torque spring 255, the spring is loaded. During dose setting, the arm 252 of the ratchet performs a dial click for each unit dialled due to the interaction with the inner teeth structure 242 of the clutch element. In the shown embodiment the clutch element is provided with 24 ratchet stops providing 24 clicks (increments) for a full 360 degrees rotation relative to the housing. The spring is preloaded during assembly which enables the mechanism to deliver both small and large doses within an acceptable speed interval. As the scale drum is rotationally engaged with the ratchet tube, but movable in the axial direction and the scale drum is in threaded engagement with the housing, the scale drum will move in a helical pattern when the dial system is turned, the number corresponding to the set dose being shown in the housing window 202.

The ratchet 252, 242 between the ratchet tube and the clutch element 240 prevents the spring from turning back the parts. During resetting, the reset tube moves the ratchet arm 252, thereby releasing the ratchet click by click, one click corresponding to one unit IU of insulin in the described embodiment. More specifically, when the dial member is turned clockwise, the reset tube simply rotates the ratchet tube allowing the arm of the ratchet to freely interact with the teeth structures 242 in the clutch element. When the dial member is turned counter-clockwise, the reset tube interacts directly with the ratchet click arm forcing the click arm towards the centre of the pen away from the teeth in the clutch, thus allowing the click arm on the ratchet to move “one click” backwards due to torque caused by the loaded spring.

To deliver a set dose, the push button 290 is pushed in the distal direction by the user as shown in FIG. 3B. The reset tube 260 decouples from the dial member and subsequently the clutch element 240 disengages the housing splines 204, whereby the strained spring is allowed to return the dial mechanism to “zero” together with the drive element 230, this leading to a dose of drug being expelled. It is possible to stop and start a dose at any time by releasing or pushing the push button at any time during drug delivery. A dose of less than 5 IU normally cannot be paused, since the rubber piston is compressed very quickly leading to a compression of the rubber piston and subsequently delivery of insulin when the piston returns to the original dimensions.

The EOC feature prevents the user from setting a larger dose than left in the cartridge. The EOC member 228 is rotationally locked to the reset tube, which makes the EOC member rotate during dose setting, resetting and dose delivery, during which it can be moved axially back and forth following the thread of the piston rod. When it reaches the proximal end of the piston rod a stop is provided, this preventing all the connected parts, including the dial member, from being rotated further in the dose setting direction, i.e. the now set dose corresponds to the remaining drug content in the cartridge.

The scale drum 270 is provided with a distal stop surface 274 adapted to engage a corresponding stop surface on the housing inner surface, this providing a maximum dose stop for the scale drum preventing all the connected parts, including the dial member, from being rotated further in the dose setting direction. In the shown embodiment the maximum dose is set to 80 IU. Correspondingly, the scale drum is provided with a proximal stop surface adapted to engage a corresponding stop surface on the spring base member, this preventing all the connected parts, including the dial member, from being rotated further in the dose expelling direction, thereby providing a “zero” stop for the entire expelling mechanism.

To prevent accidental over-dosage in case something should fail in the dialling mechanism allowing the scale drum to move beyond its zero-position, the EOC member serves to provide a security system. More specifically, in an initial state with a full cartridge the EOC member is positioned in a distal-most axial position in contact with the drive element. After a given dose has been expelled the EOC member will again be positioned in contact with the drive element. Correspondingly, the EOC member will lock against the drive element in case the mechanism tries to deliver a dose beyond the zero-position. Due to tolerances and flexibility of the different parts of the mechanism the EOC will travel a short distance allowing a small “over dose” of drug to be expelled, e.g. 3-5 IU of insulin.

The expelling mechanism further comprises an end-of-dose (EOD) click feature providing a distinct feedback at the end of an expelled dose informing the user that the full amount of drug has been expelled. More specifically, the EOD function is made by the interaction between the spring base and the scale drum. When the scale drum returns to zero, a small click arm 206 on the spring base is forced backwards by the progressing scale drum. Just before “zero” the arm is released and the arm hits a countersunk surface on the scale drum.

The shown mechanism is further provided with a torque limiter in order to protect the mechanism from overload applied by the user via the dial member. This feature is provided by the interface between the dial member and the reset tube which as described above are rotationally locked to each other. More specifically, the dial member is provided with a circumferential inner teeth structure 281 engaging a number of corresponding teeth arranged on a flexible carrier portion 261 of the reset tube. The reset tube teeth are designed to transmit a torque of a given specified maximum size, e.g. 150-300 Nmm, above which the flexible carrier portion and the teeth will bend inwards and make the dial member turn without rotating the rest of the dial mechanism. Thus, the mechanism inside the pen cannot be stressed at a higher load than the torque limiter transmits through the teeth.

Having described the working principles of a mechanical drug delivery device, embodiments of the present invention will be described.

FIG. 4 shows a switch and sensor assembly implemented in a drug delivery device of the general design described above with reference to FIGS. 2, 3A and 3B. Mainly the switch assembly per se as well as the modified members serving to actuate the individual switches are shown and will be described. In the following the structure providing contacts which are opened and closed will be denoted a switch or a switch assembly whereas a switch assembly in combination with one or more actuator or indicator members will be denoted a sensor or trigger assembly or arrangement.

More specifically, the assembly shown in FIG. 4 comprises a combined switch assembly 400, a modified housing 301, a modified ratchet tube 350, a modified clutch element 340 as well as a non-modified piston rod drive element 330. The switch assembly comprises a flexible sheet metal member 410 providing a plurality of individual flexible contact fingers, and is adapted to be mounted on a flexible printed substrate 420 on which a plurality of contact pads 428 are formed. In the shown embodiment two independent switch arrangements are incorporated which may be used in combination or independently depending on the actual configuration and design.

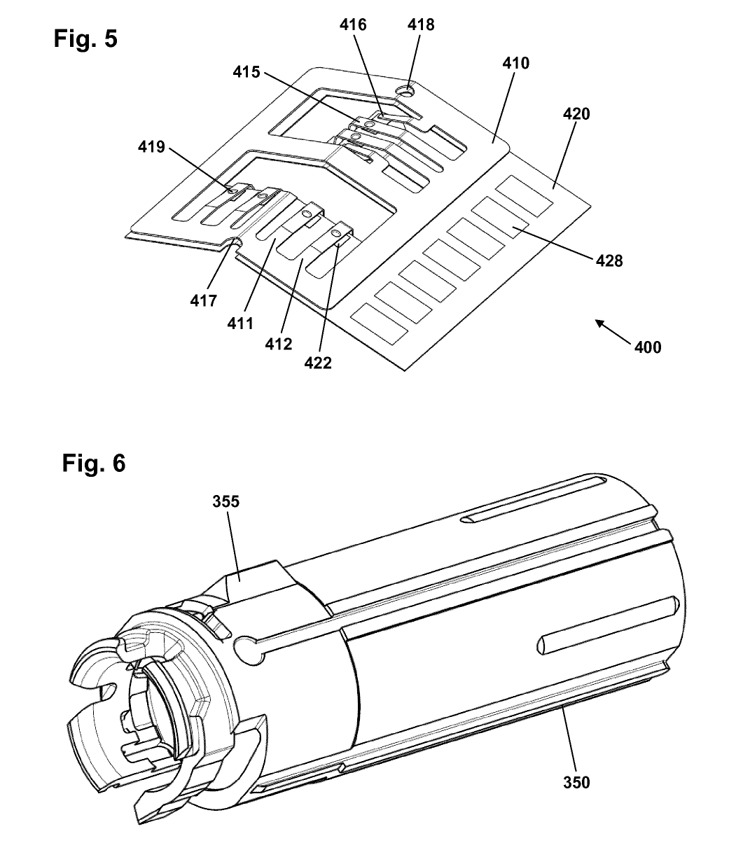

With reference to FIGS. 4 and 5 the combined switch assembly 400 will be described. The flexible substrate comprises a first opening 421 associated with a first plurality of switch contact pads (here: four) 422, as well as a second opening 424 associated with a second plurality of contact pads. In the shown embodiment the second plurality of contact pads comprises a pair of switch contact pads 425 and a pair of common ground contact pads 426. The contact pads are connected to an array of connecting contact pads 428 adapted to be connected to electronic circuitry (not shown).

The flexible sheet metal member 410 comprises a first array of flexible contact fingers (here: four) 411, each finger comprising a contact portion and an actuation portion, the contact portion comprising a contact dimple 419 adapted to engage a contact pad, and the actuation portion being adapted to engage an indicator structure. In the shown embodiment (see FIG. 5) the contact dimples are in contact with a corresponding contact pad in a resting non-actuated state (i.e. the contact is closed), whereas when the flexible arm is actuated the contact dimple is lifted out of engagement with the contact pad (i.e. the contact is opened).

The flexible sheet metal member 410 also comprises a second array of flexible contact fingers (here: two) 415, each finger comprising a contact portion and an actuation portion as described above with reference to the first array of flexible contact fingers. The second array further comprises a pair of ground contact fingers 416 adapted to be in permanent contact with corresponding ground contact pads 426 on the flexible printed substrate 420, the two fingers providing redundancy.

The switch assembly 400 is received and mounted in a recess 303 formed in the modified housing 301, the recess being provided with first and second openings 307, 308 to allow the actuation portions of the flexible contact fingers of the first respectively the second array to protrude into the interior of the housing. The recess is further provided with mounting projections 304, 305 adapted to cooperate with corresponding mounting structures 417, 418 on the switch assembly to ensure correct positioning. The switch assembly may be mounted by any suitable means, e.g. adhesive or bonding. The modified housing further comprises modified splines on the housing inner surface (not shown) adapted to cooperate with the outer spline elements 341 of the modified clutch element (see below).

Turning to the actuator or indicator elements the modified clutch element 340 is adapted to cooperate with the first array of flexible contact fingers 411 to provide a rotary sensor, whereas the modified ratchet tube 350 is adapted to cooperate with the second array of flexible contact fingers 411 to provide a trigger sensor.

The modified clutch element 340 works like the above-described clutch element 240 during dose setting and dose expelling, however, the outer spline elements 341 have been rearranged to also serve as a rotary encoder. More specifically, with the clutch element in its proximal position a given spline works as an actuator structure which lifts (when rotationally in the same position) a given contact finger to thereby keep the contact open. The rotational gap 343 between two splines is dimensioned to allow a contact finger (i.e. the neighbour finger to an actuated finger) to rest in its closed state. When the clutch element is moved to its distal position (see above) a given actuated contact finger is allowed to move down and close the corresponding contact. When the clutch element is moved proximally again at the end of an expelling event any given contact finger rotationally positioned corresponding to a spline element will be lifted and the corresponding contact opened. As appears, each time the clutch element is moved distally any open contact will be closed and each time the clutch element is moved proximally zero or more contacts will be opened. Preferably at least one contact should be closed respectively opened each time the clutch element is moved distally respectively proximally.

Depending on the number of contact fingers, the number of splines and their position, the rotary sensor can be designed to provide different input information to the associated electronic circuitry. For example with the necessary number of contact fingers and correspondingly arranged splines a rotary sensor may be designed providing an exact rotational position of the clutch element each time it is moved in and out of engagement, i.e. corresponding to the number of increments for a full rotation, e.g. 24 increments as described above with reference to FIG. 3. For a dose corresponding to less than a full rotation of the clutch element it would thus be possible to determine the dose size. Indeed, for a dose corresponding to more than a full rotation of the clutch element further sensor means would be necessary to count the number of rotations.

Although the described rotary sensor concept may be used to provide a “full” rotary position sensor, the shown embodiment is designed to provide relatively “simple” information. More specifically, the shown embodiment is designed to determine whether the clutch element during an expelling event has rotated corresponding to an even or odd number of increments.

Turning to the shown embodiment of FIGS. 4 and 5, two identical rotary sensors are provided, each comprising a pair of flexible fingers cooperating with the splines on the clutch element. Each sensor provides the same output and the two sensors thus serve to provide redundancy. The clutch element comprises 12 equidistantly arranged splines 341 with 12 correspondingly interposed gaps 343. In the following the two contact fingers of a given pair will be described as the “A” contact finger 411 and the “B” contact finger 412. The two fingers of a pair are arranged with a distance there between so that one finger is rotationally positioned corresponding to a spline with the other one positioned corresponding to a gap. Correspondingly, when the clutch element in any given rotational ratchet position is parked in its non-actuated proximal position one contact is open and one contact is closed.

The working principle for the shown embodiment can be described as follows:

In the initial axial dosing position one switch finger (e.g. the “A” finger) is lifted by a spline element serving as an actuator structure and the other (the “B” finger) is resting in a gap 343 between two actuator structures. If no dose has been set and the release button is actuated the following takes place:

1) The clutch element 340 is moved axially to the expelling position this resulting in the A-finger moving down as the lifting actuator structure is moved away, thereby closing the A-switch (closing the dimple contact point between the arm and the flex-print), this being registered by the processor. The B-finger is not moved.

2) As no dose is set the clutch element and thus the actuator structures do not rotate.

3) When the clutch element is moved axially back to the dosing position this results in the A-finger being lifted as the actuator structure is moved back, thereby opening the A-switch, this being registered by the processor. The B-finger is not moved.

As appears, in case a dose of two units was set and expelled the clutch element would rotate 2 increments resulting in each actuator structure (spline) being shifted with the neighbour member. From the perspective of the fingers there would be no difference. Correspondingly, when the same switch (A or B) is closed and subsequently opened, this is detected by the processor as an “even event”.

If a dose of one increment (e.g. 1 unit) has been set and the release button is actuated the following takes place:

1) The clutch element is moved axially to the expelling position this resulting in the A-finger moving down as the lifting actuator structure is moved away, thereby closing the A-switch, this being registered by the processor. The B-finger is not moved.

2) As a dose of one increment is set the clutch element rotates one increment during dose expelling, this resulting in the actuator structures and the gaps there between shifting position.

3) When the actuator structure is moved axially back to the dosing position this results in the B-finger being lifted up as the actuator structure is moved back, thereby opening the B-switch, this being registered by the processor. The A-finger is not moved.

As appears, the same switch actuation would take place if e.g. doses of three or five increments were set and expelled. Correspondingly, when different switches are closed and subsequently opened, this is detected by the processor as an “odd event”.

As follows from the above:

i) When 0, 2, 4 etc. increments are expelled an even event is detected.

ii) When 1, 3, 5 etc. increments are expelled an odd event is detected.

Although one switch would provide an input to the electronic circuitry for a one increment change, this would result in a single input without an on-off input which may result in uncertainty as to the movement taking place.

In a simple embodiment one switch would in theory provide the described even/odd functionality. More specifically, if a sensor assembly is provided with a single switch which initially is closed and an even number of increment is expelled then this switch may not be actuated as the actuator structures move back and forth resulting in zero switches being actuated. If an odd number of increments are expelled then this switch would be actuated only once as the actuator structures move back and forth. As appears, actuation of such a sensor system would result in either a single or no input to the electronic circuitry, which may result in uncertainty as to the movement taking place.

To ensure “positive” and safe detection of clutch movement the implementation of two switches as described above ensures an on-off input for all incremental rotational movements including no rotation.

The relevance of an even/odd event sensor will be explained below after the description of the above-mentioned trigger sensor.

As described above the modified ratchet tube 350 is adapted to cooperate with the second array of flexible contact fingers 411 to provide a trigger sensor. The modified ratchet tube 350 works like the above-described ratchet tube during dose setting and dose expelling, however, an actuator protrusion 355 has been added to serve as a trigger structure as shown in FIG. 6.

Turning to the shown embodiment of FIGS. 4-6 a trigger sensor is provided, the sensor comprising a pair of flexible contact fingers 415 and associated contact pads 425 forming two switches, the fingers being adapted to cooperate with the actuator protrusion 355. Each switch provides the same output and thus serves to provide redundancy.

As described above with reference to FIGS. 2, 3A and 3B the ratchet tube has a proximal position corresponding to a setting mode and a distal position corresponding to an expelling mode. In the embodiment of FIGS. 4-6 the trigger contact fingers 415 in a mounted state will be activated (i.e. lifted to an open switch state) by the ratchet member protrusion only when it rotates in its expelling mode. As soon as the protrusion has passed the flexible switch fingers return to a closed idle position. In this way activation of the switch is coupled to the expelling of a dose of drug whereas the user can freely set and adjust a dose without activating the switch. As the ratchet tube protrusion for larger doses passes the switch a number of times the switch will correspondingly be activated a number of times for a single expelling event, however, as the actuations take place within a very short time this just means that e.g. a timer is reset a number of times, the last reset being the one from which the time-since-last-dose is counted. Alternatively for a timer application, the first reset could be used with resets following within a short period being ignored.

The shown embodiment is designed to provide a complete open-close trigger switch activation for any expelled dose size, i.e. the ratchet tube protrusion 355 and trigger switch fingers are rotationally positioned to detect an end-of-dose event when the ratchet tube rotates from a one increment position to the initial “zero position” corresponding to a given set dose having been fully expelled. But due to tolerances and slack in the dose setting and expelling mechanism this may not always happen for the smallest possible dose size, i.e. a dose size of one increment corresponding to a rotational movement of 15 degrees of the ratchet tube for the shown embodiment.

However, when the described trigger sensor is combined with the above-described even-odd rotary sensor a combined sensor assembly is provided which with a high reliability is able to detect an expelled dose corresponding to only one increment, e.g. 1 unit of insulin.

Alternatively, if the trigger sensor is adapted to work as a rotational counter for the ratchet tube 350 and combined with a rotary sensor adapted for “full” determination of the rotational position of the clutch element a sensor assembly is provided allowing the size of an expelled dose to be determined.

As appears, the described additional rotary sensor can be provided cost-effectively without additional components being required as existing components merely have to be modified to comprise the additional sensor, i.e. the flexible sheet metal member 410, the flexible printed substrate 420 and the clutch element 340.

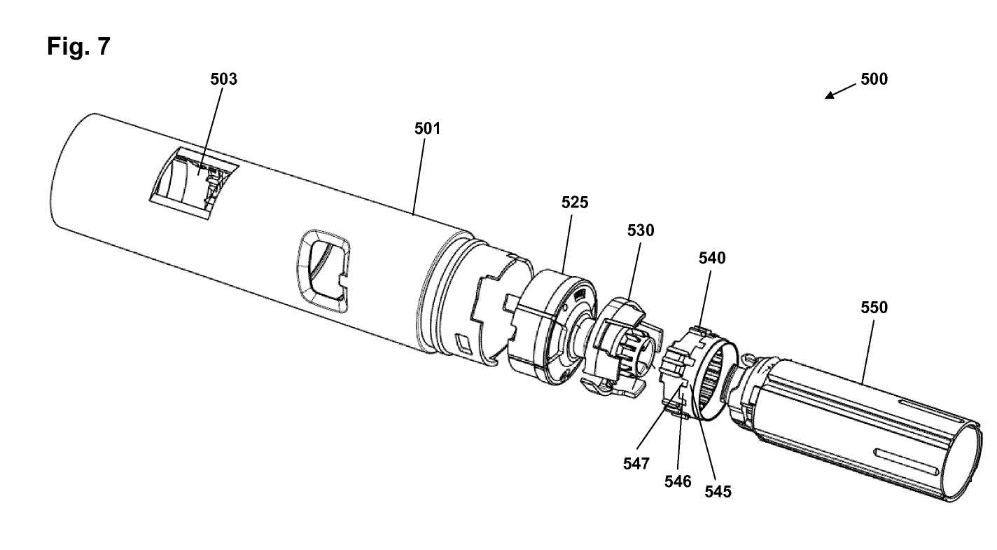

With reference to FIGS. 4-6 sensor arrangements were described based on mechanical movement of contact structures, i.e. flexible contact fingers being moved in and out of contact by mechanical structures. With reference to FIGS. 7-9 a sensor arrangement will be described based on the moving element being provided with a number of conductive structures which when moved into contact with pairs of contact fingers establish a conductive connection between the two fingers which can then be sensed by associated electronic circuitry.

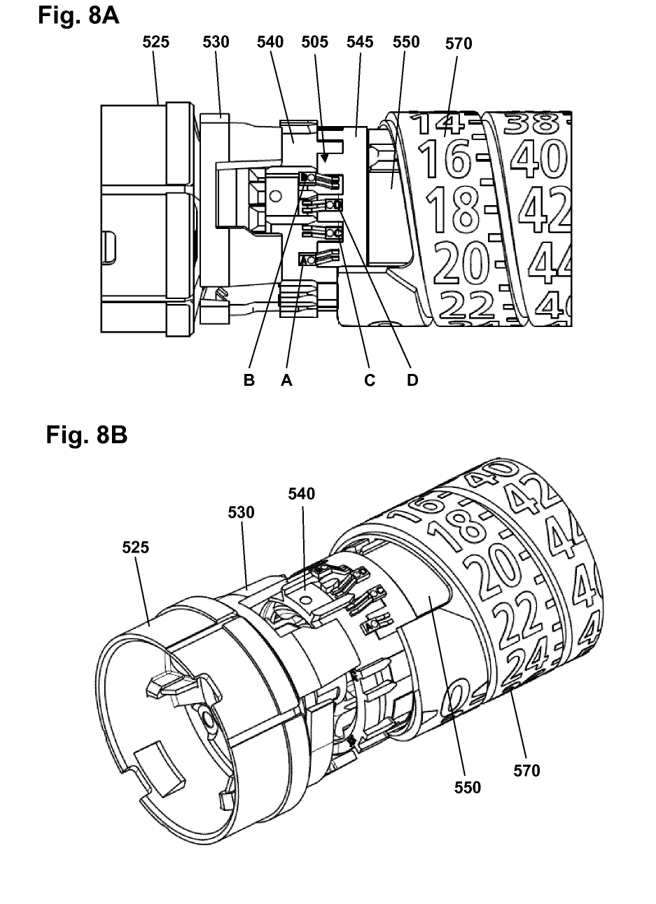

More specifically, the assembly 500 shown in FIG. 7 comprises a switch assembly with four switches (see FIG. 8A), a non-modified ratchet tube 550, a modified clutch element 540, a non-modified nut element 525 as well as a non-modified drive element 530. The clutch element is modified to comprise a circumferential proximal portion on which is arranged a conductive structure. The conductive structure comprises a proximal ring-formed portion 545 and a distal portion with a plurality of circumferentially and equidistantly arranged conductive areas 546, a plurality of non-conductive spaces 547 thereby being formed between the conductive areas. In this way a plurality of actuator structures 545, 546, 547 are formed. In the shown embodiment 12 conductive areas and 12 non-conductive spaces are provided corresponding to the 24 increments for a full rotation of the exemplary expelling mechanism. The conductive surface may be applied on the clutch element by means of printing with conductive material or by a plating process, the latter providing a more durable surface which would be relevant for a durable drug delivery device.

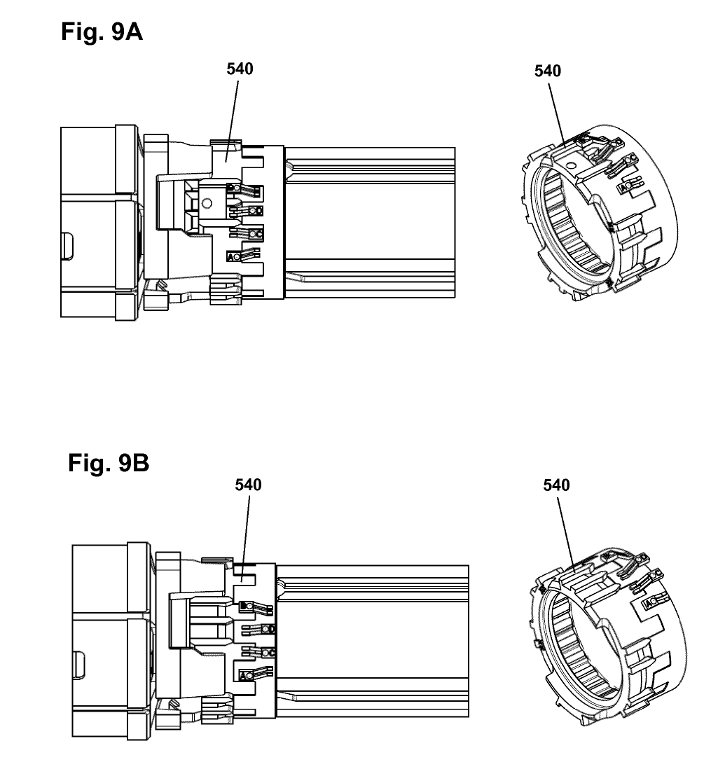

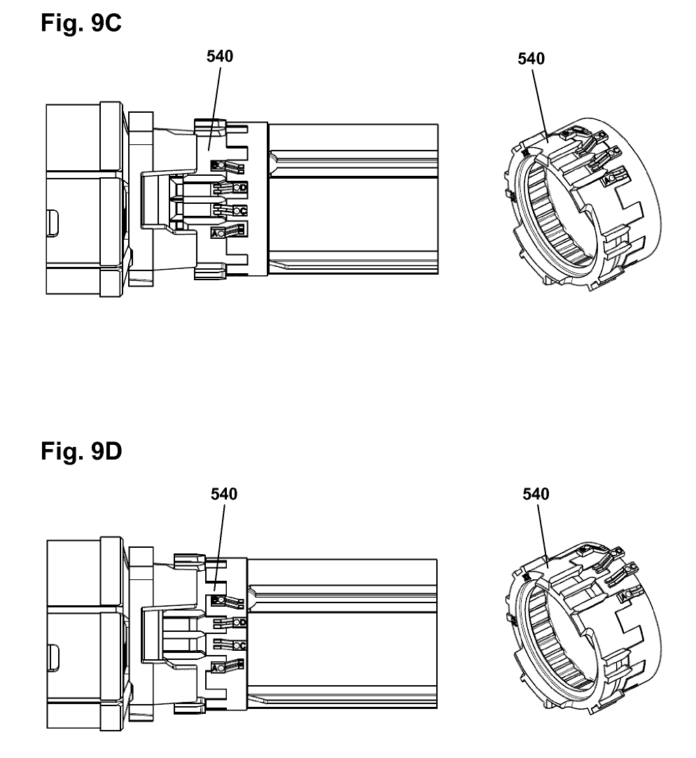

FIGS. 8A and 8B show the components of FIG. 7 in an assembled state with also the switches of the switch assembly as well as a scale drum 570 shown. The switch assembly 505 comprises four switches each having a pair of flexible contact fingers adapted to slide over the surface of the clutch element 540 as this is moved axially and/or rotated. The four switches are arranged on a carrier (not shown) which is adapted to be inserted in the housing opening 503. The switch assembly comprises a first pair of proximal switches A and B as well as a distal pair of switches C and D. As will be shown below with reference to FIGS. 9A-9D the switches A and B are positioned on the clutch element corresponding to the conductive areas 546 and non-conductive spaces 547 when the clutch element is in its proximal position, and corresponding to the conductive ring 545 when the clutch element is in its distal position. The switches C and D are positioned on the clutch element distally of the conductive areas 546 when the clutch element is in its proximal position, and corresponding to the conductive areas 546 and non-conductive spaces 547 when the clutch element is in its distal and thus rotational position. When the pair of contact fingers of a given switch is positioned on a conductive surface the switch is in a closed state “0” and when positioned on a non-conductive surface the switch is in an open state “1”, the states of the switches being detectable by associated electronic circuitry. When a given switch is actuated between the two states as the clutch element moves, this can be detected by the electronic circuitry of an associated electronic device and the information can be used for control thereof.

The switches A and B correspond to the above-described odd/even switches and are thus able to provide information whether the clutch element has rotated an odd or even number of increments between an axial actuation of the clutch element. In addition the switches A and B also supplements the switches C and D to provide a “wake up” signal when the user actuates the dose release member and thereby moves the clutch element distally (see below).

The switches C and D can be considered “transition” switches providing additional information in respect of rotation of the clutch element as well as a “wake up” signal when the user actuates the dose release member and thereby moves the clutch element distally. The wake up signal could e.g. be used to turn on the display of an associated electronic device. In the shown arrangement the switches C and D are set up to detect incremental rotation corresponding to 2 and 3 increments, e.g. corresponding to expelled doses of 2 and 3 units of insulin, this also providing an indication of 4 or more units having been expelled. As the switches C and D are positioned on the conductive areas 546 respectively the non-conductive spaces 547 when the clutch element is in its distal and thus rotational position, the switches C and D will open and close as the clutch element rotates. As each increment thus is detected the sensor arrangement may be used to provide a dose size sensor simply by counting the number of switch cycles. The sensor assembly may be combined with a further sensor system, e.g. the above-described trigger sensor operated by the ratchet tube, thereby providing additional safety for detection of small doses of expelled drug.

Turning to FIGS. 9A-9D the four different states of the switch assembly of FIG. 8A will be described.

More specifically, FIG. 9A shows the clutch element 540 in its proximal position and in a first rotational position with switch A closed, this providing the following switch states:

| A | B | C | D |
|---|---|---|---|
| 0 | 1 | 1 | 1 |

FIG. 9B shows the clutch element 540 in its distal position and in the first rotational position with switch A closed, this providing the following switch states:

| A | B | C | D |
|---|---|---|---|
| 0 | 0 | 1 | 0 |

FIG. 9C shows the clutch element 540 in its proximal position and in a second rotational position with switch A open, this providing the following switch states:

| A | B | C | D |
|---|---|---|---|
| 1 | 0 | 1 | 1 |

FIG. 9D shows the clutch element 540 in its distal position and in the second rotational position with switch A closed, this providing the following switch states:

| A | B | C | D |
|---|---|---|---|
| 0 | 0 | 1 | 0 |

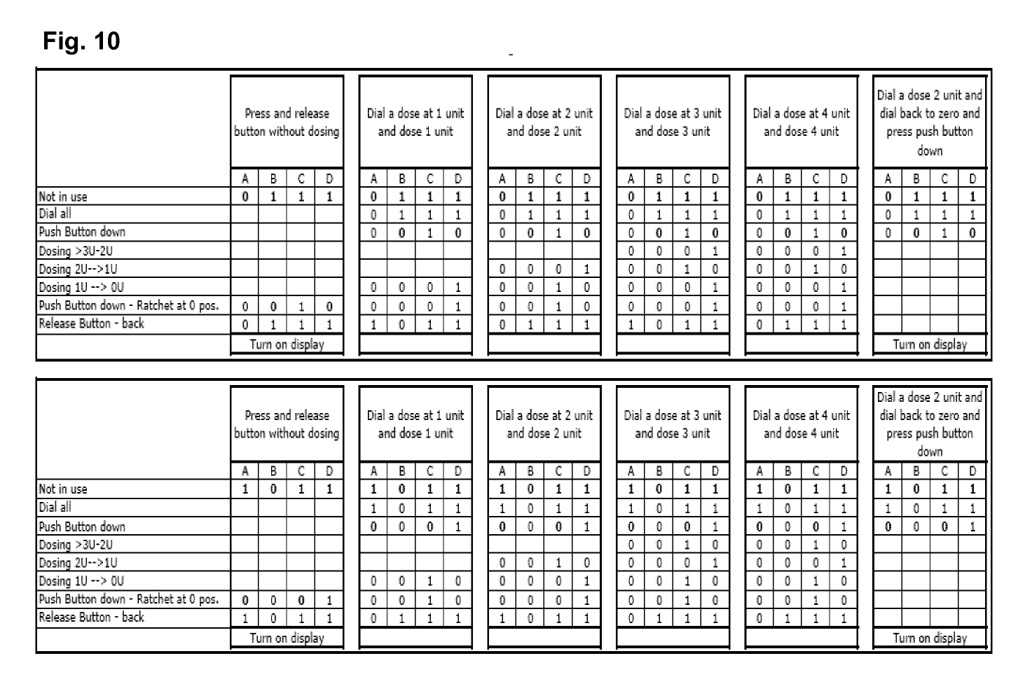

FIG. 10A illustrates in the upper table for a “closed” start state of switch A the detectable switch states as the clutch element is moved in accordance with six different user operation sequences, and in the lower table illustrates for a “closed” start state of switch B the detectable switch states as the clutch element is moved in accordance with the six different user operation sequences. The user operations are:

1) Press and release button without dosing

2) Dial a dose of 1 unit (increment) and dose 1 unit

3) Dial a dose of 2 units and dose 2 units

4) Dial a dose of 3 units and dose 3 units

5) Dial a dose of 4 units and dose 4 units

6) Dial a dose of 2 units, dial back to zero

Sequence 1 and 6 may be used to turn on a display without a dosing event and thus e.g. read out the current status of e.g. time since last dose. Movement between two “framed” boxes provides a “wake-up” signal”. As indicated, when an expelled dose is detected, the associated electronics may be reset, e.g. a counter for a time-since-last-dose timer.

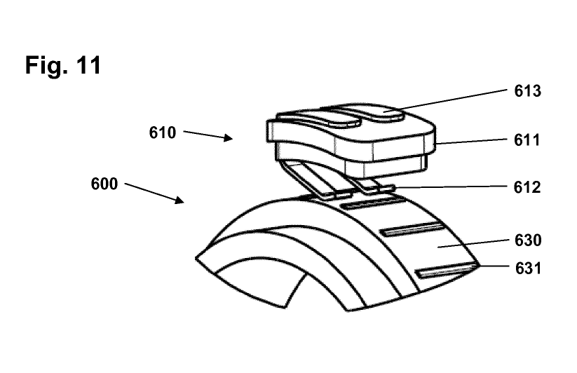

With reference to FIGS. 4-10 sensor arrangements are described which detects rotation of a component having a proximal position corresponding to a setting mode and a distal position corresponding to an expelling mode. With reference to FIG. 11 a further sensor arrangement will be described which detects rotation of a component of the expelling mechanism which is not moved axially but only rotates during expelling of a dose.

FIG. 11 shows a switch and sensor assembly 600 implemented in a drug delivery device of the general design described above with reference to FIGS. 2, 3A and 3B. Mainly the switch assembly per se as well as the modified indicator member serving to actuate the switch are shown and will be described.

More specifically, FIG. 11 shows a sensor assembly 600 comprising a stationary switch assembly 610 in combination with a rotatable drive element 630 serving as an indicator member, the latter in the form of a modified piston rod drive element, the un-modified element being described above with reference to FIGS. 2, 3A and 3B. The switch assembly comprises a carrier 611 adapted to be mounted in a corresponding recess in a correspondingly modified housing (not shown), a pair of flexible conductive contact fingers 612 each connected to a contact pad 613 adapted to be connected to further electronic circuitry. As in the FIG. 8A embodiment the sensor arrangement of FIG. 11 is not based on mechanical movement of contact structures, but based on the moving element being provided with a number of conductive structures which when moved into contact with the contact fingers establishes a conductive connection between the two fingers and thus actuates the switch from an open to a closed state, and subsequently from a closed to an open state when the conductive structure is moved out of contact with the contact fingers which can then be sensed by associated electronic circuitry. More specifically, the drive element 630 comprises on its outer circumference a number of actuator structures in the form of circumferentially arranged axially oriented conductive “stripe” structures 631 which when positioned corresponding to the two contact fingers will establish electrical contact there between. Between the conductive structures non-conductive “gaps” are provided by the non-conductive polymeric material from which the drive element 630 is manufactured. The conductive stripes may be provided by any suitable means, e.g. by printing with conductive ink.

As appears, when the drive element 630 rotates during expelling of a dose the switch assembly 610 is closed and shortly after opened again each time a conductive stripe 631 passes the contact fingers 612, this indicating to the associated electronics that an expelling event is taking place. In the shown embodiment the number of conductive stripes, and thus interposed gaps, correspond to the number of increments for a full rotation, e.g. 24 increments. As each increment thus is detected the sensor arrangement may be used to provide a dose size sensor simply by counting the number of switch cycles, or the sensor arrangement may be used as a simple trigger sensor to simply detect that an expelling event has taken place irrespective of whether 5, 10 or 25 cycles have been detected.

The stripes are positioned on the drive element 630 such that for a given “parked” rotational position of the drive element the pair of fingers are arranged in the middle between two stripes, this providing a robust design for detecting a rotational movement of only one increment. However, due to tolerances and slack in the expelling mechanism this may not always happen for the smallest possible dose size, i.e. a dose size of one increment corresponding to a rotational movement of 15 degrees of the drive element 630 for the shown embodiment.

However, when the described trigger sensor is combined with the above-described even-odd rotary sensor a combined sensor assembly is provided which with a high reliability is able to detect an expelled dose corresponding to only one increment, e.g. 1 unit of insulin.

In an alternative embodiment (not shown) the switch assembly 610 in FIG. 11 may be in the form of a mechanical switch assembly of the type described with reference to FIG. 5, with the drive element modified to have a number of circumferentially arranged axially oriented rib-like protrusions serving to move one or more flexible contact fingers to thereby open and close the associated contact. As for the trigger sensor described with reference to FIGS. 5 and 6 two contact fingers may be provided for redundancy.

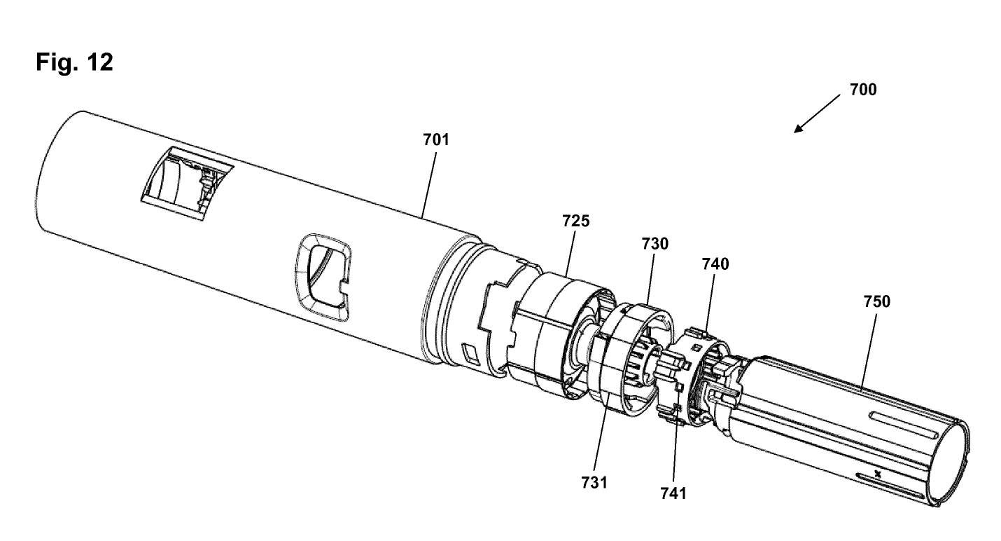

FIG. 12 shows an assembly 700 comprising components adapted to cooperate with two switch assemblies, the assembly comprising a modified housing 701, a non-modified ratchet tube 750, a modified clutch element 740, a modified nut element 725 as well as a modified drive element 730. The drive element is modified to comprise a circumferential proximal portion on which is arranged a plurality of axially oriented conductive structures 731 adapted to cooperate with a switch assembly (not shown) of the type shown in FIG. 11. The clutch element 740 is modified to comprise a circumferential proximal portion comprising a plurality of indicator structures 741 (here: openings) adapted to cooperate with a switch assembly (not shown) of the type shown in FIG. 5. The nut element 725 is modified merely to accommodate the modified drive element 730.

In the shown embodiment the drive element 730 comprises 12 circumferentially and equidistantly arranged axially oriented conductive structures. As the expelling mechanism has 24 increments for a full rotation the “resolution” of the sensor system will only ensure that an expelled dose corresponding to 2 increments is detected. Correspondingly, a second sensor system is provided adapted for detection of rotation corresponding to an odd or even number of increments, this corresponding to the above-described sensor system with the difference that the actuator structures are in form of openings instead of splines.

The above-described sensor assembly as well as the individual components may be used to provide input to associated electronic circuitry in different forms via the output contact pads 428. For example, if the sensor assembly is incorporated in a durable drug delivery device the electronic circuitry may be in the form of individual “traditional” electronic components mounted on e.g. a flexible printed circuit board (PCB) and may provide more advanced features like a memory for storing data for a number of expelling events (e.g. dose size and/or dose time), wired or wireless connectivity, traditional LCD or OLED, as well as an exchangeable or rechargeable power source.

For a disposable drug delivery device the same kind of traditional electronic circuitry may be provided in the form of an add-on module adapted to be mounted on a first device and subsequently transferred to another device by the user, typically when the cartridge has been emptied, the add-on module being provided with contact terminals for engagement with the output contact pads 428.

Whereas “traditional” electronics in most cases would be considered too expensive for incorporation in a disposable drug delivery device, an integrated solution may be provided using “alternative” technologies allowing for cost-effective manufacturing.

Correspondingly, in the following a drug delivery device will be described comprising a flexible “electronic label” based fully or in part on “printed electronics” and adapted to be permanently mounted on the curved exterior of a drug delivery device, the label comprising contact terminals adapted to cooperate with the above-described output contact pads 428. The electronic label is designed to provide the user with information in respect of time-since-last-dose in a simple and intuitive way. A detailed description of a possible design and manufacturing process for such a label can be found in EP2014/074475 which is hereby incorporated by reference. In the following merely the user-oriented functionality of such a label will be described.

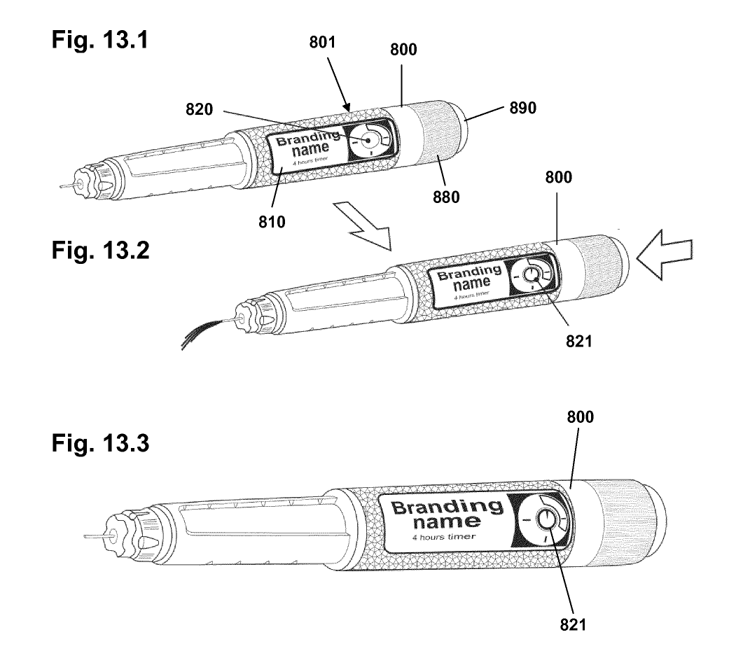

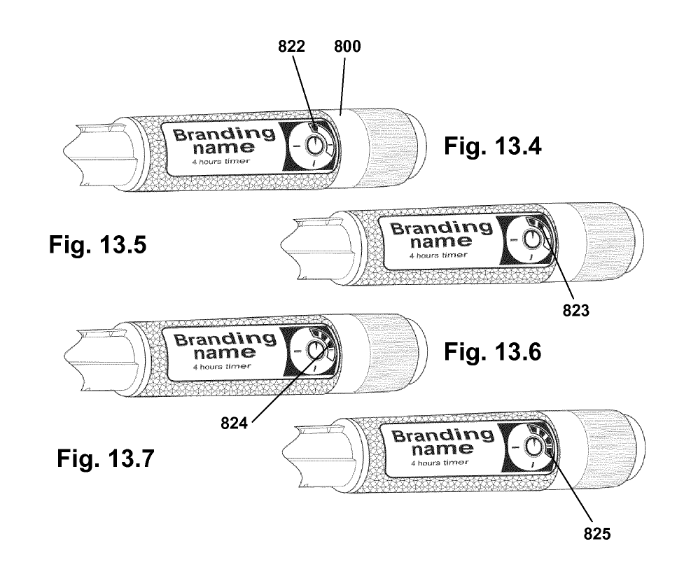

Turning to FIGS. 13.1-13.7 a drug delivery device 800 of the type described with reference to FIGS. 1-12 and comprising one the above-described sensor arrangements is provided with an electronic label 801. The electronic label is formed on a flexible foil 810 and comprises a printed display 820, a mounted chip, a mounted battery, an array of contact terminals for attachment to the sensor assembly output pads, and a plurality of printed leads connecting terminals of the different components. The label has a generally rectangular form with a cutout (not to be seen) specific for the drug delivery device for which the label is intended allowing the display window 202 to be viewed.

With reference to FIGS. 13.1-13.7 use of the finalized pen-formed drug delivery device will be described. The user receives the pen with the label in an inactive or sleeping state with all segments of the display in an “off” state, which would not change during setting of a dose by rotating the dose setting member 880 (FIG. 13.1). Thus, if the pen was reset to zero after a dose had been set and put away, the pen would remain in the inactive state. Correspondingly, if the release button 890 is actuated with no dose set the pen will remain inactive. When a dose has been set and the user releases the expelling mechanism to expel a dose, the ratchet tube 350 will be moved distally and start to rotate together with the clutch element 340, this resulting in triggering of the sensor assembly and subsequently actuation of the label timer, this turning on a central timer symbol 821 on the label (FIG. 13.2). The pen label will remain in this state for an hour (FIG. 13.3) after which a counting symbol 822 will be activated (FIG. 13.4) with a further counting symbol 823, 824, 825 being activated for each subsequent hour as shown in FIGS. 13.5-13.7, i.e. after 2, 3 and 4 hours after delivery of a dose of drug. For the shown embodiment, after 5 hours all segments will be de-activated.

Corresponding to the above description of different sensor designs, dose size sensing and dose expelling event sensing may be combined. Indeed, if dose size related information is to be displayed on the electronic label, corresponding numeric display means should be provided. In addition to a set dose and/or the size of the last set dose, also the remaining amount of drug in the cartridge could be displayed.

The above-described electronic label could be provided with additional features or the electronic label could be used as a platform to provide a drug delivery with further features. For example, manufacturers of insulin products often make different types of insulin, some of which are working rapidly but not for very long and others that works slower, but for longer time. As a further example a temperature sensor may be provided. The measured temperature may e.g. be used as an input for calculating a variable expiration date or warn against exposure to excessive temperatures. In addition to the above-described display features a logging functionality may be provided, e.g. a display graphically illustrating when drug was expelled, e.g. day and/or time. Warnings may be provided against e.g. double doses, maximum dose exceeded or other abnormal use. Personal settings may be entered wirelessly via e.g. an NFC antenna. The display means of the electronic label may be adapted to display 2D matrix codes which can be used to transfer data to e.g. a smartphone provided with a camera.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A rotary sensor assembly, comprising:
an indicator member adapted to rotate in increments and having a rotationally locked first axial position and a rotationally free second axial position,
input structure adapted to be actuated, directly or indirectly, by movement of the indicator member, and
a processor adapted to receive input from the input structure,
wherein:
the indicator member comprises a plurality of actuator structures,
the input structure comprises one or more switches adapted to be actuated by an actuator structure,
zero or more switches is/are actuated when the indicator member is moved from the first to the second axial position, this corresponding to a first switch pattern,
zero or more switches is/are actuated when the indicator member is moved from the second to the first axial position, this corresponding to a second switch pattern, and
the processor is adapted to determine incremental rotational movement of the indicator member based on input from the one or more switches corresponding to the first and second switch patterns.

2. A rotary sensor assembly as in claim 1, wherein:
at least one switch is actuated when the indicator member is moved from the first to the second axial position, this corresponding to a first switch pattern, and
at least one switch is actuated when the indicator member is moved from the second to the first axial position, this corresponding to a second switch pattern.

3. A rotary sensor assembly as in claim 1, wherein the actuator structures and switch(es) are arranged to provide first and second switch patterns allowing the processor to determine whether the indicator member has rotated corresponding to a an even or odd number of increments.

4. A rotary sensor assembly as in claim 3, comprising first and second switches, the actuator structures being arranged on the indicator member such that:
for a given rotational position the first switch only is actuated by an actuator structure when the indicator member is moved from the first to the second axial position, this corresponding to the first switch pattern, and
for a rotational movement of an odd number of increments the second switch only is actuated when the indicator member is moved from the second to the first axial position, this corresponding to the second switch pattern.

5. A drug delivery device, comprising:
a housing having an exterior surface,
a rotary sensor assembly as in claim 1,
a drug-filled cartridge or structure for receiving a drug-filled cartridge, the cartridge comprising an outlet and an axially displaceable piston,
drug expelling structure comprising:
dose setting structure allowing a user to set a dose amount of drug to be expelled in increments,
a piston rod adapted to engage and axially move the piston to thereby expel an amount of drug from the cartridge through the outlet,
the indicator member,
a sensor system comprising:
the input structure adapted to be actuated, directly or indirectly, by movement of the indicator member,
the processor adapted to receive input from the input structure, and
an energy source,
wherein:
the indicator member is arranged to rotate during expelling of a dose, the amount of rotation corresponding to a number of increments,
the indicator member is in the first axial position when the drug expelling structure is in a dose setting state, and in the second axial position when the drug expelling structure is in an expelling state,
the indicator member comprises a plurality of actuator structures,
the input structure comprises one or more switches adapted to be actuated by an actuator structure,
zero or more switches is/are actuated when the indicator member is moved from the first to the second axial position, this corresponding to a first switch pattern,
zero or more switches is/are actuated when the indicator member is moved from the second to the second axial position, this corresponding to a second switch pattern, and
the processor is adapted to determine incremental movement of the indicator member based on input from the one or more switches corresponding to the first and second switch patterns.

6. A drug delivery device as in claim 5, wherein:
at least one switch is actuated when the indicator member is moved from the first to the second axial position, this corresponding to a first switch pattern,
at least one switch is actuated when the indicator member is moved from the second to the first axial position, this corresponding to a second switch pattern,

7. A drug delivery device as in claim 5, wherein the actuator structures and switch(es) are arranged to provide first and second switch patterns allowing the processor to determine whether the indicator member during expelling of a dose has rotated corresponding to a an even or odd number of increments.

8. A drug delivery device as in claim 7, comprising first and second switches, the actuator structures being arranged on the indicator member such that:
for a given rotational position the first switch only is actuated by an actuator structure when the indicator member is moved from the first to the second axial position, this corresponding to the first switch pattern, and
for a rotational movement of an odd number of increments the second switch only is actuated when the indicator member is moved from the second to the first axial position, this corresponding to the second switch pattern.

9. A drug delivery device as in claim 5, further comprising:
a second indicator member arranged to move during expelling of a dose,
a second sensor system comprising:
second input structure adapted to be actuated, directly or indirectly, by movement of the second indicator member, and
the processor adapted to receive input from the second input structure.

10. A drug delivery device as in claim 9, wherein:
the second indicator member is adapted to rotate from a set position corresponding to a set dose amount and to an end-of-dose position in which the set dose has been expelled,
the second indicator member has a first axial position when the drug expelling structure is in a dose setting state, and a second axial position when the drug expelling structure is in an expelling state, and
the second input structure is actuated when the second indicator member has reached the end-of-dose position when the second indicator member is in the second axial position.

11. A drug delivery device as in claim 9, wherein:
the second indicator member is adapted to rotate during expelling from an initial position to an end-of-dose position in which the set dose has been expelled, the amount of rotation corresponding to the expelled dose amount.

12. A drug delivery device as in claim 5, comprising a display adapted to display a time parameter, wherein:
the processor is adapted to, based on input from the input structure, control the display to display a time parameter related to the time the input structure was actuated.

13. A drug delivery device as in claim 12, comprising a flexible sheet on which is formed or mounted:
the display adapted to display a time parameter,
the processor, and
the energy source,
wherein the flexible sheet is mounted at least in part to the exterior of the housing.

14. A drug delivery device as in claim 13, wherein at least one of the display, processor, and energy source is/are in the form of printed electronics.
